(19) United States
(12) Patent Application Publication
(10) Pub. No.: US 2023/0374209 A1
(43) Pub. Date: Nov. 23, 2023

(54) SYSTEMS AND METHODS FOR GENERATING A HYDROGEL FROM A CO2 GAS STREAM

(71) Applicant: SHELL OIL COMPANY, Houston, TX (US)

(72) Inventors: Nicoleta Cristina NENU, Amsterdam (NL); Jieheng GUO, Amsterdam (NL); Andreas KLEMT, Leuna (DE); Sven SCHOLZ, Leuna (DE)

(21) Appl. No.: 18/042,613

(22) PCT Filed: Sep. 29, 2021
(86) PCT No.: PCT/EP2021/076835

§ 371 Date: Feb. 23, 2023

(30) Foreign Application Priority Data
Sep. 29, 2020 (EP) 20198962.1
Oct. 2, 2020 (EP) 20199944.8

(51) Int. Cl. C08G 64/30; B01J 23/44; B01J 27/053

(52) U.S. Cl. CPC C08G 64/305; B01J 23/44; B01J 27/053; C08G 2210/00

(57) ABSTRACT

The present disclosure relates to a method for generating a hydrogel from a CO₂ gas stream. The method for converting a CO₂ gas stream comprising a CO₂ into an ester, comprises the conversion of CO2 into a (COOH)2 preferably by passing the CO2 through a water bath to produce a carbonated water; and passing the carbonated water through a metal ion exchange bubble column comprising a M₂(COO)₂ to produce the (COOH)₂ and a MHCO₃; reacting the (COOH)₂ with a mono-alcohol to obtain the ester. The invention further relates to a system for converting CO2.

### FIELD OF THE DISCLOSURE

The present disclosure relates, in some embodiments, systems and methods that convert CO₂ from a CO₂ stream (e.g., produced by an oil and gas industry asset) into a value product including a polymer in the form of a hydrogel.

### BACKGROUND OF THE DISCLOSURE

Our lives depend on energy as a major contributor for our daily survival. A large portion of the energy we use is derived from the processing and combustion of fossil fuels (e.g., hydrocarbon fuels). However, besides generating energy, these combustion processes also generate greenhouse gases that are believed to be a leading cause of global climate change.

Two main tactics are used deal with anthropogenic emissions of greenhouse gases. The first tactic is to simply find ways to reduce the overall fossil fuel consumption through energy use limitation or use of alternative energy methods. However, since energy is necessary, this tactic does little to deal with the greenhouse gases that will still be generated. A second option is to instead substantially transform the generated greenhouse gases into environmentally benign or even beneficial products. This greenhouse gas abatement technology enables fossil fuel consumption without regulating fossil fuel consumption.

Carbon dioxide (CO₂) is the primary greenhouse gas. There are currently existing technologies that capture and store CO₂ as it is generated from a fossil fuel processing or combustion system, but these technologies do not lead to the generation of beneficial products. There is a need for viable CO₂ gas abatement technologies that not only substantially capture the CO₂ once it is formed but then subsequently transform it into a useful commercially and environmentally beneficial products.

Additionally, besides adding to global climate change, the escape of terrestrial carbon as the backbone of CO₂ represents a lost opportunity to maintain adequate carbon levels in soil to promote crop growth. Therefore, not only is the loss of CO₂ damaging the atmosphere, but it is reducing access to a necessary soil resource. There is also a need to develop methods in which to increase soil carbon and protect the soil against loss of soil carbon. The two needs described above are not mutually exclusive and may be solved in tandem.

### SUMMARY

Accordingly, there is a need for improved methods and systems for forming a hydrogel from a CO₂ gas stream. The method for converting a CO2 gas stream comprises a CO2 into an ester, the method comprising:
converting the CO2 into a (COOH)2 preferably by passing the CO2 through a water bath to produce a carbonated water;
passing the carbonated water through a metal ion exchange bubble column comprising a M2(COO)2 to produce the (COOH)2 and a MHCO3; and
reacting the (COOH)2 with a mono-alcohol to obtain the ester.

In some embodiments, a method may include a step of converting CO₂ into (COOH)₂ preferably by passing a CO₂ gas stream including a CO₂ through a water bath to produce a carbonated water. A carbonated water may be passed through a metal ion exchange bubble column including a M₂(COO)₂ to produce a (COOH)₂ and a MHCO₃. A method may include combining a glycerine, an acid catalyst comprising a H₂SO₄, and one or more of a (COOCH₃)₂ and a (COOEt)₂ produced from the metal ion exchange bubble column to produce a hydrogel and a methanol or ethanol. In some embodiments, a method includes a step of combining the MHCO₃ produced from a metal ion exchange bubble column with a hydrogen gas in a hydrogenation reactor comprising a palladium catalyst at a temperature range of about 15 to about 150° C. and a pressure of about 0.1 bara to about 100 bara to produce a MHCO₃ and HCOOM mixture and a step of separating the MHCO₃ and HCOOM mixture through fractional crystallization in a crystallization unit into a separated MHCO₃ that may be recycled back to the hydrogenation reactor and a separated HCOOM. A method may include treating a HCOOM to an inert thermal treatment in dryer/inert treatment reactor with a catalytic amount of MOH at a temperature ranging from about 100° C. to about 400° C. to produce a hydrogen gas that may be transferred to a hydrogenation reactor and a dried M₂(COO)₂ that may be transferred to the metal ion exchange bubble column. In some embodiments, M=Na (sodium) and K (potassium). According to some embodiments, a M₂(COO)₂ may include one or more of K₂(COO)₂ and Na₂(COO)₂. A HCOOM may include one or more of HCOOK and HCOONa. A MHCO₃ may include one or more of NaHCO₃ and NaHCO₃. A MOH may include one or more of KOH and NaOH.

In some embodiments the method may include a step of passing a (COOH)₂ through an activated carbon bed to produce a (COOH)₂ absorbed carbon bed comprising an absorbed (COOH)₂ and passing an alcohol comprising one or more of a methanol and an ethanol through the (COOH)₂ absorbed carbon bed to produce a one or more of a (COOCH₃)₂ and a (COOEt)₂.

According to some embodiments, the present disclosure relates to system for manufacturing an ester from a CO2 gas stream comprising a CO2, the system comprising: a CO2 conversion unit configured to convert the CO2 into a (COOH)2; a metal ion exchange bubble column to produce the (COOH)2 and a MHCO3; and a reactor for reacting the (COOH)2 with a mono-alcohol to obtain an ester; preferably the reactor comprises an activated carbon bed configured to receive the (COOH)2 and a mono-alcohol to generate the ester.

According to some embodiments, the present disclosure relates to a system for generating a hydrogel from a CO₂ gas stream. A system may include a metal ion exchange bubble column that may be connected to a CO₂ gas stream source through a CO₂ gas inlet, to a polymerization unit through a (COOEt)₂ transfer line, to a dryer/inert treatment reactor through a M₂(COO)₂ transfer line, and a hydrogenation reactor through a MHCO₃ transfer line. A metal ion exchange bubble column may be configured to combine a CO₂ gas stream containing a CO₂ with a M₂(COO)₂ to produce a (COOEt)₂ and a MHCO₃. A system may include a polymerization unit that may be configured to receive a (COOEt)₂ from a metal ion exchange bubble column through a (COOEt)₂ transfer line and to combining a glycerine, an acid catalyst comprising a H₂SO₄, and the (COOEt)₂ to produce a hydrogel and a methanol or ethanol.

In some embodiments, a system may include a hydrogenation reactor that may be configured to receive a MHCO₃ from a metal ion exchange bubble column through a MHCO₃ transfer line, the hydrogenation reactor connected to a crystallization unit through a MHCO₃/HCOOM mixture transfer line. A hydrogenation reactor may be configured to combine the MHCO₃ with a hydrogen gas and a palladium catalyst at a temperature range of about 15 to about 150° C. and a pressure range from about 0.1 bara to about 100 bara to produce a MHCO₃ and HCOOM mixture. A system may include a crystallization unit that may be configured to receive a MHCO₃ and HCOOM mixture through a MHCO₃/HCOOM mixture transfer line. A crystallization unit may be connected to a dryer/inert treatment reactor through a HCOOM transfer line and the crystallization unit may be configured to separate a MHCO₃ and HCOOM mixture through fractional crystallization into a separated MHCO₃ and a separated HCOOM. In some embodiments, a system may include a dryer/inert treatment reactor that may be configured to receive a separated HCOOM from a crystallization unit and to treat a HCOOM to an inert thermal treatment with a catalytic amount of KOH at a temperature ranging from about 100° C. to about 400° C. to produce a hydrogen gas and a dried M₂(COO)₂.

The present disclosure relates, according to some embodiments, to a use of a hydrogel produced from a CO₂ gas stream to sequester carbon in soils. A use may include the use comprising the step of combining a hydrogel with a soil. A hydrogel may be formed by various steps as disclosed herein.

According to some embodiments, a method for generating a hydrogel from a CO₂ gas stream includes passing the CO₂ gas stream containing a CO₂ through an absorption column with a MOH to produce a MHCO₃ and an off gas and a step of combining the MHCO₃ produced from the metal ion exchange bubble column with a H₂ in a hydrogenation reactor including a catalyst at a temperature ranging from about 15° C. to about 150 ° C. and a pressure ranging from about 0.1 bara to about 100 bara to produce a HCOOM. In some embodiments, a method may include treating a HCOOM to an inert thermal treatment in a dryer/inert treatment reactor 125 with a catalytic amount of a MOH at a temperature ranging from about 100° C. to about 400° C. to produce a H₂ that may be transferred to a hydrogenation reactor and a dried M₂(COO)₂ that may be transferred to the metal ion exchange bubble column. A method may include combining M₂(COO)₂ with a Ca(OH)₂ in a reactive multi-stage forced cooling crystallization system to produce a Ca(COO)₂ and a MOH and a step of combining the Ca(COO)₂ with a H₂SO₄ in a reactive crystallization system to produce a (COOH)₂ and a CaSO₄. A method may include a step of combining a (COOH)₂, a CH₃CH₂OH, and a first acid catalyst containing a H₂SO₄ in a reactive distillation reactor at a temperature ranging from about 80° C. to about 100° C. and under atmospheric pressure to produce a (COOEt)₂. A method may include combining a (COOEt)₂, a glycerine, and a second acid catalyst including a H₂SO₄ in a reactive distillation reactor at a temperature of about 160° C. and a pressure ranging from about 0.3 bara to about 1 bara to produce a hydrogel and an ethanol.

### BRIEF DESCRIPTION OF THE DRAWINGS

Some embodiments of the disclosure may be understood by referring, in part, to the present disclosure and the accompanying drawings, wherein:

FIG. 1 is a diagram of system for generating a hydrogel from a CO₂ gas stream, according to a specific example embodiment of the disclosure;

FIG. 2 is a diagram of system for generating a hydrogel from a CO₂ gas stream including a off gas tank, a CO Convertor, and a N2, CₓH_{y} collector, according to a specific example embodiment of the disclosure;

FIG. 3 is a plot showing water uptake and release cycles from 600 hours to 2,800 hours, according to a specific example embodiment of the disclosure; and

FIG. 4 is a plot showing a water uptake cycle from 0 hours to 800 hours, according to a specific example embodiment of the disclosure.

### DETAILED DESCRIPTION

The present disclosure relates, in some embodiments, to systems and methods for manufacturing an ester from a CO₂ gas stream. A disclosed system and method advantageously and directly converts CO₂, a greenhouse gas, into a hydrogel, which may be used to increase and protect existing soil carbon levels. Additionally, disclosed systems and methods do this in a scalable and less complex manner in comparison to existing chemical synthetic technologies that also create additional environmentally harmful waste products. Additionally, many of the method steps and system components involve catalytic cycles that recycle byproducts from other methods steps and system components, thereby minimizing waste-products.

### Hydrogel

According to some embodiments, a hydrogel includes a polymer (e.g., polyester) made from a polymerization reaction performed by combining a glycerine and a (COOEt)₂ in the presence of an acid catalyst (e.g., H₂SO₄). A disclosed polymer may have a structure according to Formula I:

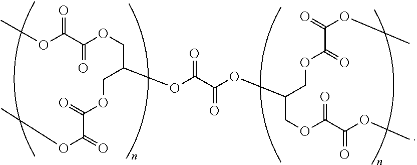

A hydrogel according to Formula I may include an n value ranging from about 1 to about 5,000. In some embodiments, a hydrogel according to Formula I may include a n value of about 1, or of about 500, or of about 1,000, or of about 1,500, or of about 2,000, or of about 2,500, or of about 3,000, or of about 3,500, or of about 4,000, or of about 4,500, or of about 5,000, where about includes plus or minus 250.

### Systems for Converting a CO₂ to a Hydrogel

According to some embodiments, as shown in FIG. 1, a disclosed system 100 for converting a CO₂ to a hydrogel may include a metal ion exchange bubble column 110 connected to a CO₂ gas stream 105 through a CO₂ gas transfer line, to a hydrogenation reactor 115 through a MHCO₃ transfer line and a EtOH transfer line, to a polymerization reactor 130 through a (COOMe)₂ transfer line, and to a dryer/inert treatment reactor 125 through a M₂(COO)₂ transfer line. In some embodiments, a hydrogenation reactor 115 may be connected to a H₂ tank 145 through a H₂ transfer line, to a crystallization unit 120 through both a MHCO₃/MOOCH transfer line and a MHCO₃ transfer line, and to a dryer/inert treatment reactor 125 through a H₂ transfer line. A crystallization unit 120 may be connected to a dryer/inert treatment reactor 125 through a MOOCH transfer line. A polymerization reactor may be connected to a 2nd reactant stream 135 through a 2nd reactant transfer line and may be connected to a hydrogel tank 140 through a hydrogel transfer line.

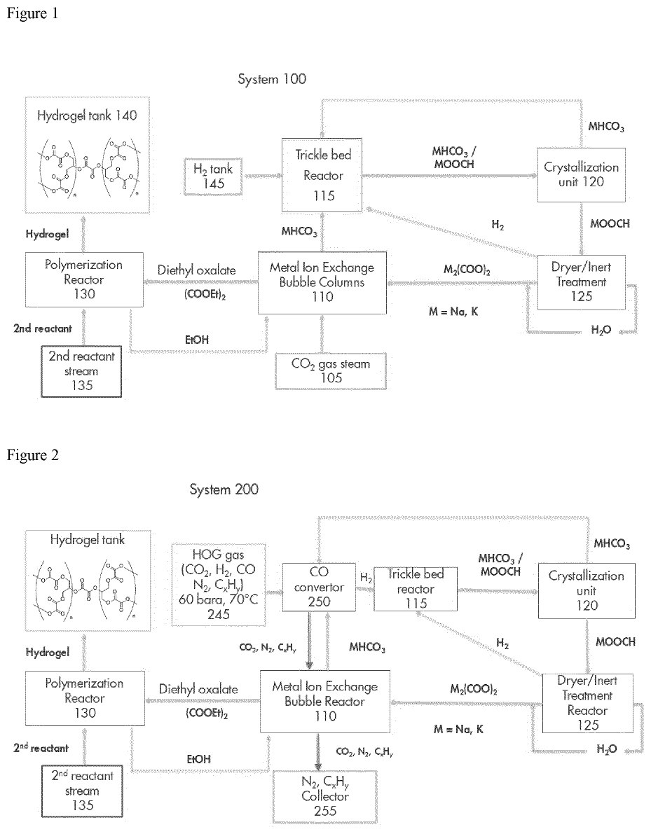

In some embodiments, as shown in FIG. 2, a system 200 includes an off gas tank 245, a CO convertor 250, and a N₂, CₓH_{y} Collector 255. A CO converter 250 may be connected to an off gas tank 245 through an off gas transfer line, connected to a crystallization unit 120 through a MHCO₃ transfer line, connected to a metal ion exchange bubble column 110 through both a MHCO₃ transfer line and a CO₂, N₂, CₓH_{y} transfer line, and connected to a hydrogenation reactor 115 through a hydrogenation reactor transfer line.

As shown in FIGS. 1 and 2, a system may include a CO₂ gas stream 105, which may be provided by a tank or streamed from a CO₂ gas source such as a device containing a fuel combustion or processing component. A CO₂ gas stream 105 may be connected to a metal ion exchange bubble column 110 through a CO₂ gas transfer line and may configured to transfer a CO₂ gas through the CO₂ gas transfer line. Within a metal ion exchange bubble column 110, a CO₂ gas may be combined with a base including one or more of KOH and NaOH to produce one or more of a KHCO₃ and a NaHCO₃. A metal ion exchange bubble column 110 includes a bubble column including a vertically arranged or horizontally arranged column of any shape and size. In some embodiments, a CO₂ gas transfer line may provide a CO₂ gas at a bottom, at a top, or in a position between the bottom and the top of a bubble column. For example, a metal ion exchange bubble column 110 may be configured to receive a CO₂ gas from a CO₂ gas transfer line at a position of a bubble column found in the lower half of the bubble column. An ion exchange bubble column 110 may be at a temperature ranging from about 5° C. to about 60° C. For example, an ion exchange bubble column 110 may be at a temperature of about 5° C., or about 10° C., or about 15° C., or about 20° C., or about 25° C., or about 30° C., or about 35° C., or about 40° C., or about 45° C., or about 50° C., or about 55° C., or about 60° C., where about includes plus or minus 2.5° C. In some embodiments, an ion exchange bubble column 110 may produce one or more of a MHCO₃, which may precipitate at a bottom of the ion exchange bubble column 110. A solution containing a MHCO₃ may be transferred from an ion exchange bubble column 110 to a CO convertor 250 through a MHCO₃ transfer line.

In some embodiments, as shown in FIG. 1, a system 100 may include a hydrogenation reactor 115 containing a catalyst including one or more of a palladium catalyst, a nickel catalyst, and a platinum catalyst. A catalyst includes, but is not limited to, a Pd/C, a Pd/Al₂O₃, a Ni/SiO₂, a Pd/theta Al₂O₃, a SiO₂/Al2O₃, and combinations thereof. A method includes using a catalyst at a concentration ranging from about 0.1 g/100 mL of solvent (e.g., organic or inorganic) to about 5 g/100 mL of solvent. A method includes using a catalyst at a concentration of about 0.1 g/100 mL of solvent, or about 0.5 g/100 mL of solvent, or about 1.0 g/100 mL of solvent, or about 1.5 g/100 mL of solvent, or about 2.0 g/100 mL of solvent, or about 2.5 g/100 mL of solvent, or about 3.0 g/100 mL of solvent, or about 3.5 g/100 mL of solvent, or about 4.0 g/100 mL of solvent, or about 4.5 g/100 mL of solvent, or about 5.0 g/100 mL of solvent, where about includes plus or minus 0.25 g/100 mL of solvent. As shown in FIG. 1, a hydrogenation reactor 115 may be connected to a H₂ tank 145 through a H₂ transfer line, to a crystallization unit 120 through both a MHCO₃/MOOCH transfer line and a MHCO₃ transfer line, and to a dryer/inert treatment reactor 125 through a H₂ transfer line. In some embodiments, a hydrogenation reactor 115 may be configured to receive a MHCO₃ from a metal ion exchange bubble column and to combine the MHCO₃ with a hydrogen gas and a palladium catalyst at a temperature ranging from about of 15° C. to about 150° C. and a pressure ranging from about 0.1 bara to about 100 bara to produce a MHCO₃ and HCOOM mixture. A hydrogenation reactor 115 contains a MHCO₃ at a concentration ranging from about 0.5 M to about 5 M. A hydrogenation reactor 115 may contain a MHCO₃ at a concentration of about 0.5 M, or about 1.0 M, or about 1.5 M, or about 2.0 M, or about 2.5 M, or about 3.0 M, or about 3.5 M, or about 4.0 M, or about 4.5 M, or about 5.0 M, where about includes plus or minus 0.25 M. In some embodiments, a hydrogenation reactor 115 may contain from about 50 mL to about 1,000 mL of a MHCO₃ solution. In some embodiments, a liquid space velocity inside a hydrogenation reactor 115 includes a range from about 0.1 v/vh to about 5 v/vh. A hydrogenation reactor 115 may include a temperature of about 15° C., or about 20° C., or about 25° C., or about 30° C., or about 35° C., or about 40° C., or about 45° C., or about 50° C., or about 55° C., or about 60° C., or about 65° C., or about 70° C., or about 75° C., or about 80° C., or about 85° C., or about 90° C., or about 95° C., or about 100° C., or about 105° C., or about 110° C., or about 115° C., or about 120° C., or about 125° C., or about 130° C., or about 135° C., or about 140° C., or about 145° C., or about 150° C., where about includes plus or minus 2.5° C. According to some embodiments, a hydrogenation reactor 115 may include a hydrogen pressure of about 0.001 bara, or about 0.005 bara, or about 0.01 bara, or about 0.05 bara, or about 0.1 bara, or about 0.5 bara, or about 1.0 bara, where about includes plus or minus 0.0025 bara in between 0.001 bara and 0.01 bara, plus or minus 0.025 in between 0.01 and 0.1 bara, and plus or minus 0.25 in between 0.1 bara and 1.0 bara. A disclosed method may include a hydrogen pressure of about 1.0 bara, or about 10 bara, or about 20 bara, or about 30 bara, or about 40 bara, or about 50 bara, or about 60 bara, or about 70 bara, or about 80 bara, or about 90 bara, or about 100 bara, where about includes plus or minus 5 bara. In some embodiments, in place of a hydrogenation reactor 115, a system may include a stirred-tank reactor including a suspended catalyst at a concentration ranging from about 0.01 g/L to about 100 g/L. A stirred-tank reactor may include a suspended catalyst at a concentration of about 0.01 g/L, or about 0.1 g/L, where about includes plus or minus 0.05 g/L. A stirred-tank reactor may include a suspended catalyst at a concentration of about 1.0 g/L, or about 10 g/L, or about 20 g/L, or about 30 g/L, or about 40 g/L, or about 50 g/L, or about 60 g/L, or about 70 g/L, or about 80 g/L, or about 90 g/L, or about 100 g/L, where about includes plus or minus 5 g/L.

In some embodiments, as shown in FIG. 1, once a hydrogenation reactor forms a MHCO₃ and HCOOM mixture, the mixture may be transferred to a crystallization unit 120 through a MHCO₃/HCOOM transfer line. A crystallization unit 120 may be configured to receive a MHCO₃ and HCOOM mixture through a MHCO₃/HCOOM mixture transfer line. In some embodiments, a crystallization unit 120 may be connected to a dryer/inert treatment reactor 125 through a MOOCH transfer line. A crystallization unit may be configured to separate a MHCO₃ and HCOOM mixture through fractional crystallization into a separated MHCO₃ and a separated HCOOM. In some embodiments, a crystallization unit 120 includes a container made of any material (e.g., glass, metal, plastic), a cooling element, a heating element (e.g., thermocouple), and a vacuum).

In some embodiments, as shown in FIG. 1, a HCOOM (e.g., HCOONa, HCOOK) produced by a crystallization unit 120 may be transferred to a dryer/inert treatment reactor 125 through a HCOOM transfer line. A dryer/inert treatment reactor 125 may be configured to treat a HCOOM to an inert thermal treatment with a catalytic amount of a MOH (e.g., KOH, NaOH) at a temperature ranging from about 300° C. to about 400° C. to produce a dried M₂(COO)₂. A catalytic amount of a MOH may include a range from about 1 wt % MOH to about 5 wt % MOH (wt % relative to the amount of HCOOM and MOH). In some embodiments, a dryer/inert treatment reactor 125 may be configured to treat a HCOOM to an inert thermal treatment at a temperature of about 300° C., or about 310° C., or about 320° C., or about 330° C., or about 340° C., or about 350° C., or about 360° C., or about 370° C., or about 380° C., or about 390° C., or about 400° C., or about 410° C. where about includes plus or minus 5° C. A dryer/inert treatment reactor 125 may include a container made of any substance (e.g., metal, glass, plastic), a heating element (e.g., thermocouple), and a feed line for a MOH.

As shown in FIG. 1, a disclosed system may include a polymerization reactor 130 connected to a metal ion exchange bubble column 110 and may receive a (COOMe)₂ from the metal ion exchange bubble column 110 through a (COOMe)₂ transfer line. In some embodiments, a polymerization reactor 130 may receive one or more of a (COOMe)₂ and a (COOEt)₂ from a bubble reactor 110 and convert it to a hydrogel as well. A polymerization reactor 130 may be connected to a 2nd reactant stream 135 through a 2nd reactant transfer line and may be connected to a hydrogel tank 140 through a hydrogel transfer line. In some embodiments, a polymerization reactor 130 may combine a (COOMe)₂ with a glycerine supplied by a 2nd reactant stream 135 and a catalytic amount of an acid (e.g., H₂SO₄) to produce a hydrogel and an ethanol. An acid includes any known acid including nitric acid, sulfuric acid, perchloric acid, chloric acid, acetic acid, sulfurous acid, methanoic acid, phosphoric acid, nitrous acid, hydrofluoric acid, para-toluenesulfonic acid and combinations thereof. A catalytic amount of acid includes form about 0.01 wt. % to about 1.0 wt. %, by weight of the one or more of a (COOMe)₂ and a (COOEt)₂. A catalytic amount of an acid includes 0.01 wt. %, or about 0.05 wt. %, or about 0.1 wt. %, or about 0.25 wt. %, or about 0.5 wt. %, or about 0.75 wt. %, or about 1.0 wt. %, where about includes plus or minus 0.05 from 0.01 wt. % to 0.1 wt. % and plus or minus 0. 125 from 0.1 wt. % to 1.0 wt. %. A polymerization reactor 130 may include a reaction container made from any material (e.g., a glass, a metal, a plastic) that is made of any general size (e.g., lab, pilot plant, plant) and shape, a heating element, a cooling element, a vacuum element, a mixing element (e.g., stirrer, shaker), and a pressure regulator.

As shown in FIG. 1, a system 100 may include a 2nd reactant stream 135. A 2nd reactant stream 135 may include a container made from any know material (e.g., a glass, a plastic, a metal) that may be an open container or a closed container. A 2nd reactant stream 135 may include any conveyance mechanism to facilitate a transfer of a 2nd reactant (e.g., glycerine) from a 2nd reactant stream 135 to a polymerization reactor 130 through a 2nd reactant transfer line.

In some embodiments, a system 100 includes a hydrogel tank 140 that may be configured to receive a hydrogel from a polymerization reactor 130 through a hydrogel transfer line. A hydrogel tank 140 may include a container made from any know material (e.g., a glass, a plastic, a metal) that may be an open container or a closed container.

According to some embodiments, as shown in FIG. 2, a system may include a off gas tank 245 configured to transfer a gas including a CO₂, a H₂, a CO, a N₂, and a hydrocarbon having the formula CₓH_{y}, from the off gas tank 245 to a CO converter 250 through a off gas transfer line. In some embodiments, a off gas tank 245 may be maintained at a pressure ranging from about 40 bara to about 70 bara and at a temperature ranging from about 50° C. to about 80° C. A off gas tank 245 may include a container made from any know material (e.g., a glass, a plastic, a metal).

In some embodiments, a system 200 as shown in FIG. 2 includes a CO convertor 250, which may be configured to receive a off gas from a off gas tank 245 and to transform the off gas into an H₂ and a gas including a CO₂, a N₂, and a hydrocarbon having the formula CₓH_{y}. A CO Convertor 250 may produce an H₂ and may transfer it to a hydrogenation reactor 115 through an H₂ transfer line. In some embodiments, a CO Convertor 250 may produce a gas including a CO₂, a N₂, and a hydrocarbon having the formula CₓH_{y} and may transfer the gas to a metal ion exchange bubble column 110 through a CO₂, N₂, CₓH_{y} transfer line. In some embodiments, a CO convertor 250 may receive a MHCO₃ from a metal ion exchange bubble column 110 through a MHCO₃ transfer line. As shown in FIG. 2, a system 200 may include a N₂, CO₂, CₓH_{y} collector 255. A N₂, CO₂, CₓH_{y} collector 255 may be connected to a metal ion exchange bubble column 110 and may be configured to receive a gas including a N₂, a CO₂, and a CₓH_{y} from the metal ion exchange bubble column 110 through a CO₂, N₂, CₓH_{y} transfer line. A N₂, CO₂, CₓH_{y} collector 255 may include a container made from any material include a plastic, a glass, and a metal and may be of any size or shape. A N₂, CO₂, CₓH_{y} collector 255 may include an open container and a closed container. In some embodiments, a N₂, CO₂, CₓH_{y} collector 255 may include a transfer line (e.g., pipe) that may convey gas away from system 200. For example, a N₂, CO₂, CₓH_{y} collector 255 may include including a pipe line and a vehicle (e.g., transfer truck).

### Methods for Converting a CO₂ to a Hydrogel

The present disclosure, according to some embodiments, relates to a method for generating a formic acid from a CO₂ gas stream. An exemplary pathway includes the stoichiometry shown below:
1) KOH+CO2→KHCO3
2) KHCO3+H2→HCOOK+H2O
3) HCOOK+H-IE→HCOOH+K-IE
4) 2HCOOH+2EtOH→Et2(COO)2+2H2O
5) aEt2(COO)2+bCH2OH—CHOH—CH2OH→CxHyOz+cEtOH

### Alternatively

1) KOH+CO2→KHCO3
2) KHCO3+H2→HCOOK+H2O
3) 2HCOOK+Ca(OH)2→Ca(COO)2+2KOH
4) Ca(COO)2+H2SO4→CaSO4+H2(COO)2
5) aEt2(COO)2+bCH2OH—CHOH—CH2OH→CxHyOz+cEtOH
6) 5OAEt2+2Gly→10EtOH+5H₂O+[Gly(OA)₂]OA

An exemplary pathway disclosed herein includes a method using a metal ion exchange bubble column 110 to convert a CO₂ gas into a (COOMe)₂, using a polymerization reactor 130 to convert the (COOMe)₂ to a hydrogel, using a hydrogenation reactor 115 to convert a MHCO₃ to a MHCO₃/MOOCH mixture, a crystallization unit 120 to convert the MHCO₃/MOOCH mixture to a HCOOM, and a dryer/inert treatment reactor 125 to convert the HCOOM to a M₂(COO₂) that is fed to the metal ion exchange bubble column to be converted to the (COOMe)₂. According to some embodiments, the present disclosure relates to a method for generating a hydrogel from a CO₂ gas stream. A method may include a step of passing a CO₂ gas stream including a CO₂ through a water bath to produce a carbonated water. A water bath may be cooled or heated. In some embodiments, a water bath may be set at a temperature ranging from about 0° C. to about 100° C. A water bath may be set at a temperature of about 0° C., or about 10° C., or about 20° C., or about 30° C., or about 40° C., or about 50° C., or about 60° C., or about 70° C., or about 80° C., or about 90° C., or about 100° C., where about includes plus or minus 5° C. A carbonated water may be from about 1% saturated with a CO₂ to about 100% saturated with a CO₂. A carbonated water may be about 1% saturated with a CO₂, or about 10% saturated with the CO₂, or about 20% saturated with the CO₂, or about 30% saturated with the CO₂, or about 40% saturated with the CO₂, or about 50% saturated with the CO₂, or about 60% saturated with the CO₂, or about 70% saturated with the CO₂, or about 80% saturated with the CO₂, or about 90% saturated with the CO₂, or about 100% saturated with the CO₂, where about includes plus or minus 5% saturation.

According to some embodiments, a carbonated water may be passed through an ion exchange bubble column 110 including a M₂(COO)₂ (e.g., Na₂(COO)₂, K₂(COO)₂) to produce a (COOH)₂ and a MHCO₃ (e.g., NaHCO₃, KHCO₃). An ion exchange column may be acidic or basic. In some embodiments, a method may include a step of passing a (COOH)₂ generated in an ion exchange bubble column 110 through an activated carbon bed to produce a (COOH)₂ absorbed carbon bed. In some embodiments, a (COOH)₂ absorbed carbon bed may be from about 1% saturated with absorbed (COOH)₂ to about 100% saturated with absorbed (COOH)₂. A (COOH)₂ absorbed carbon bed may be about 1% saturated with absorbed (COOH)₂, or about 10%, or about 20%, or about 30%, or about 40%, or about 50%, or about 60%, or about 70%, or about 80%, or about 90%, or about 100%, where about includes plus or minus 5%.

In some embodiments, a method may include a step of passing a mono-alcohol through a (COOH)₂ absorbed carbon bed to produce a one or more of a (COOCH₃)₂, a (COOEt)₂, and any alcohol based product based on the alcohol passed through the (COOH)₂ absorbed carbon bed. For example, if ethanol is used a (COOEt)₂ may be generated or if methanol is used a (COOCH₃)₂ may be generated. In some embodiments, a mono-alcohol includes methanol, ethanol, propanol, any straight chain or branched alcohol C₁-C₁₀, and combinations thereof.

According to some embodiments, a method may include forming one or more of a (COOCH₃)₂, a (COOEt)₂, and other alcohol reaction formed products by combining a (COOH)₂, an alcohol (e.g., methanol, ethanol, and a first acid catalyst (e.g., H₂SO₄) in a reactive distillation reactor at a temperature ranging from about 80° C. to about 100° C. and under atmospheric pressure. An acid includes any known acid including hydrochloric acid, nitric acid, sulfuric acid, hydrobromic acid, hydroiodic acid, perchloric acid, chloric acid, acetic acid, sulfurous acid, methanoic acid, phosphoric acid, nitrous acid, hydrofluoric acid, and combinations thereof. A catalytic amount of acid includes form about 0.01 wt. % to about 1.0 wt. %, by weight of the one or more of a (COOMe)₂ and a (COOEt)₂. A catalytic amount of an acid includes 0.01 wt. %, or about 0.05 wt. %, or about 0.1 wt. %, or about 0.25 wt. %, or about 0.5 wt. %, or about 0.75 wt. %, or about 1.0 wt. %, where about includes plus or minus 0.05 from 0.01 wt. % to 0.1 wt. % and plus or minus 0.125 from 0.1 wt. % to 1.0 wt. %. In some embodiments, an alcohol includes methanol, ethanol, propanol, any straight chain or branched alcohol C₁-C₁₀, and combinations thereof. A temperature includes about 80° C., or about 85° C., or about 90° C., or about 95° C., or about 100° C., where about includes plus or minus 2.5° C.

In some embodiments, a method may include combining a glycerine, an acid catalyst (e.g., a H₂SO₄), and one or more of a (COOCH₃)₂ and the (COOEt)₂ produced from the metal ion exchange bubble column 110 to produce a hydrogel and an alcohol (e.g., methanol, ethanol). An acid includes any known acid including hydrochloric acid, nitric acid, sulfuric acid, hydrobromic acid, hydroiodic acid, perchloric acid, chloric acid, acetic acid, sulfurous acid, methanoic acid, phosphoric acid, nitrous acid, hydrofluoric acid, and combinations thereof. A catalytic amount of acid includes form about 0.01 wt. % to about 1.0 wt. %, by weight of the one or more of a (COOMe)₂ and a (COOEt)₂. A catalytic amount of an acid includes 0.01 wt. %, or about 0.05 wt. %, or about 0.1 wt. %, or about 0.25 wt. %, or about 0.5 wt. %, or about 0.75 wt. %, or about 1.0 wt. %, where about includes plus or minus 0.05 from 0.01 wt. % to 0.1 wt. % and plus or minus 0. 125 from 0.1 wt. % to 1.0 wt. %. In some embodiments, a method may include removing the alcohol by-product through treatment with heat, a vacuum, or both to produce a substantially alcohol-free hydrogel. In some embodiments, a produced hydrogel does not need to be substantially alcohol free.

In some embodiments, a method may include a step of combining a MHCO₃ produced from an ion exchange bubble column 110 with a hydrogen gas in a hydrogenation reactor 115 including a catalyst (e.g., a palladium catalyst) at a temperature ranging from about 15° C. to about 100° C. and a pressure ranging from about 0.1 bara to about 100 bara to produce a HCOOM and a MHCO₃ mixture. A method may include a catalyst including one or more of a palladium catalyst, a nickel catalyst, and a platinum catalyst. A catalyst includes, but is not limited to, a Pd/C, a Pd/Al₂O₃, a Ni/SiO₂, a Pd/theta Al₂O₃, a SiO₂/Al2O₃, and combinations thereof. A method includes using a catalyst at a concentration ranging from about 0.1 g/100 mL of solvent (e.g., organic or inorganic) to about 5 g/100 mL of solvent. A method includes using a catalyst at a concentration of about 0.1 g/100 mL of solvent, or about 0.5 g/100 mL of solvent, or about 1.0 g/100 mL of solvent, or about 1.5 g/100 mL of solvent, or about 2.0 g/100 mL of solvent, or about 2.5 g/100 mL of solvent, or about 3.0 g/100 mL of solvent, or about 3.5 g/100 mL of solvent, or about 4.0 g/100 mL of solvent, or about 4.5 g/100 mL of solvent, or about 5.0 g/100 mL of solvent, where about includes plus or minus 0.25 g/100 mL of solvent. A hydrogenation reactor 115 may include a temperature of about 15° C., or about 20° C., or about 25° C., or about 30° C., or about 35° C., or about 40° C., or about 45° C., or about 50° C., or about 55° C., or about 60° C., or about 65° C., or about 70° C., or about 75° C., or about 80° C., or about 85° C., or about 90° C., or about 95° C., or about 100° C., or about 105° C., or about 110° C., or about 115° C., or about 120° C., or about 125° C., or about 130° C., or about 135° C., or about 140° C., or about 145° C., or about 150° C., where about includes plus or minus 2.5° C. According to some embodiments, a hydrogenation reactor 115 may include a hydrogen pressure of about 0.001 bara, or about 0.005 bara, or about 0.01 bara, or about 0.05 bara, or about 0.1 bara, or about 0.5 bara, or about 1.0 bara, where about includes plus or minus 0.0025 bara in between 0.001 bara and 0.01 bara, plus or minus 0.025 in between 0.01 and 0.1 bara, and plus or minus 0.25 in between 0.1 bara and 1.0 bara. A disclosed method may include a hydrogen pressure of about 1.0 bara, or about 10 bara, or about 20 bara, or about 30 bara, or about 40 bara, or about 50 bara, or about 60 bara, or about 70 bara, or about 80 bara, or about 90 bara, or about 100 bara, where about includes plus or minus 5 bara.

In some embodiments, a method may include a step of separating a MHCO₃ and HCOOM mixture through fractional crystallization in a crystallization unit 120 into a separated MHCO₃ (e.g. NaHCO₃, KHCO₃) and a separated HCOOM (e.g., HCOONa, HCOOK). A separated MHCO₃ may be recycled back to a hydrogenation reactor 115 and a separated HCOOM may be transferred to a dryer/inert treatment reactor 125. A crystallization unit 120 may be at a temperature ranging from about 0° C. to about 500° C. A temperature includes about 0° C., or about 50° C., or about 100° C., or about 150° C., or about 200° C., or about 250° C., or about 300° C., or about 350° C., or about 400° C., or about 450° C., or about 500° C., where about includes plus or minus 25° C. A temperature of a crystallization unit 120 may be maintained by a cooling jacket, a cooling bath, a heating jacket, a thermocouple, or any other means of temperature control. Within a crystallization unit 120, any solvent may be used including both organic (e.g., methanol, ethyl acetate, hexanes, methylene chloride) and aqueous (e.g., water) solvents. A solvent may be added to a crystallization unit from a solvent tank. According to some embodiments, no solvents are used. In some embodiments, a crystallization unit 120 may be made of any material including metal, glass, ceramic, plastic, and combinations thereof. A crystallization unit 120 may separate a MHCO₃ (e.g. NaHCO₃, KHCO₃) and a HCOOM by turning one into a solid while one remains a liquid or by forming separate solids of each.

According to some embodiments, a method may include a step of treating a HCOOM to an inert thermal treatment in a dryer/inert treatment reactor 125 with a catalytic amount of a MOH (e.g., KOH, NaOH) at a temperature of ranging from about 100° C. to about 400° C. to produce a hydrogen gas and a dried M₂(COO)₂ (e.g., K₂(COO)₂, Na₂(COO)₂. A temperature includes about 100° C., or about 120° C., or about 140° C., or about 160° C., or about 180° C., or about 200° C., or about 220° C., or about 240° C., or about 260° C., or about 280° C., or about 300° C., or about 320° C., or about 340° C., or about 360° C., or about 380° C., or about 400° C., where about includes plus or minus 10° C. In some embodiments, a hydrogen gas may be transferred to a hydrogenation reactor 115 and a dried M₂(COO)₂ may be transferred to an ion exchange bubble column 110. A catalytic amount of a MOH may include the MOH at a concentration ranging from about 0.01 mmol to about 1 mmol. A MOH concentration includes about 0.01 mmol, or about 0.1 mmol, or about 0.2 mmol, or about 0.3 mmol, or about 0.4 mmol, or about 0.5 mmol, or about 0.6 mmol, or about 0.7 mmol, or about 0.8 mmol, or about 0.9 mmol, or about 1 mmol, where about includes plus or minus 0.05 mmol. A MOH includes NaOH, KOH, and NH₄OH, and combinations thereof and preferably NaOH and/or KOH. An inert thermal treatment may be conducted in the presence of an inert gas including nitrogen, argon, helium, and combinations thereof. An inert gas may be introduced into a dryer/inert treatment reactor 125 from one or more inert gas tanks through a gas pressure regulator. In some embodiments, a method includes combining a M₂(COO)₂ with a MOH in a reactive multi-stage forced cooling crystallization system to produce a M₂(COO)₂ and a MOH. A M₂(COO)₂ may be combined with an acid (e.g., H₂SO₄) in a reactive crystallization system to produce a (COOH)₂ and a M₂SO₄. A reactive crystallization system may be at a temperature ranging from about 40° C. to about −200° C. A temperature includes about 40° C., or about 20° C., or about 0° C., or about −20° C., or about −40° C., or about −60° C., or about −80° C., or about −100° C., or about −120° C., or about −140° C., or about −160° C., or about −180° C., or about −200° C., where about includes plus or minus 10° C. A temperature of a reactive crystallization system may be maintained by a cooling jacket, a cooling bath, a heating jacket, a thermocouple, or any other means of temperature control. Within a reactive crystallization system, any solvent may be used including both organic (e.g., methanol, ethyl acetate, hexanes, methylene chloride) and aqueous (e.g., water) solvents. A solvent may be added to a reactive crystallization system from a solvent tank. According to some embodiments, no solvents are used. In some embodiments, a reactive crystallization system may be made of any material including metal, glass, ceramic, plastic, and combinations thereof. A reactive crystallization system may separate a (COOH)₂ and a M₂SO₄ by turning one into a solid while one remains a liquid or by forming separate solids of each.

In some embodiments, a method includes a step of passing a CO₂ gas stream including a CO₂ through an absorption column including a MOH (e.g., NaOH, KOH) to produce a MHCO₃ (e.g., NaHCO₃, KHCO₃) and an off gas. A separated MHCO₃ may be recycled back to a hydrogenation reactor 115 and a separated HCOOM.

The present disclosure further relates, according to some embodiments, to methods of using a hydrogel produced from a CO₂ gas stream to sequester carbon in soils. A hydrogel may be combined with a soil to produce a soil-based hydrogel product. In some embodiments, a bentonite may be added to a soil-based hydrogel products at a weight ranging from about 1 wt. % to about 50 wt. %, by weight of the soil-based hydrogel product, which may produce a dry powder. A soil-based hydrogel product may include a bentonite at about 1 wt. %, or about 10 wt. %, or about 20 wt. %, or about 30 wt. %, or about 40 wt. %, or about 50 wt. %, where about includes plus or minus 5 wt. %, by weight of the soil-based hydrogel product. A formed powder product may desirably be formed into tablets, pellets, and other shapes and sizes.

According to some embodiments, a disclosed method for generating an ester from a CO₂ gas stream includes a step of converting a CO₂ from the CO₂ gas stream into a (COOH)₂, and combining the (COOH)₂, a CH₃CH₂OH, and a first acid catalyst comprising a H₂SO₄ at a temperature ranging from about 80° C. to about 100° C. and under atmospheric pressure to produce the carbon sequestering agent containing a (COOEt)₂. The obtained ester may be considered an intermediate product and a carbon sequestering agent as it is obtained from carbon monoxide.

A disclosed method may include a step of combining an ester, a glycerine, and a second acid catalyst (e.g., H₂SO₄) at a temperature ranging from about 100° C. to about 200° C. to produce a poly-ester, preferably in the form of hydrogel, and an ethanol. A temperature includes about 100° C., or about 110° C., or about 120° C., or about 130° C., or about 140° C., or about 150° C., or about 160° C., or about 170° C., or about 180° C., or about 190° C., or about 200° C., where about includes plus or minus 5° C. In some embodiments, a disclosed method may include a step of combining an ester, a glycerine, and a second acid catalyst at a pressure ranging from about 0.1 bara to about 100 bara to produce a polyester which preferably in the form of a hydrogel, and an ethanol. A pressure includes about 0.001 bara, or about 0.005 bara, or about 0.01 bara, or about 0.05 bara, or about 0.1 bara, or about 0.5 bara, or about 1.0 bara, where about includes plus or minus 0.0025 bara in between 0.001 bara and 0.01 bara, plus or minus 0.025 in between 0.01 and 0.1 bara, and plus or minus 0.25 in between 0.1 bara and 1.0 bara. A disclosed method may include a hydrogen pressure of about 1.0 bara, or about 10 bara, or about 20 bara, or about 30 bara, or about 40 bara, or about 50 bara, or about 60 bara, or about 70 bara, or about 80 bara, or about 90 bara, or about 100 bara, where about includes plus or minus 5 bara.

In a disclosed embodiment the polyester is in the form of a hydrogel.

Disclosed embodiments also include methods of supplementing a soil with a carbon sequestering agent, such as a hydrogel, as described herein. A method may include a step of combining at least a hydrogel with a soil, where the hydrogel comprises a polyester which includes one or more of a (COOCH₃)₂ and a (COOEt)₂. A hydrogel may have a structure according to Formula I:

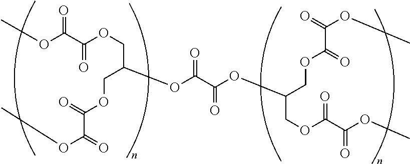

In some embodiments, a disclosed polyester hydrogel may decompose into CO₂, water, small organic building blocks (e.g., organic carboxylic acids), and other environmentally benign products. Decomposition of a polyester hydrogel may be facilitated by microbes (e.g., bacteria, fungi), heat, water, sunlight, weather, cold, acids provided by the environment (e.g., acid rain), and combinations thereof. In some embodiments, a polyester hydrogel may autonomously decompose. A disclosed polyester hydrogel may retain, absorb, or release carbon dioxide while decomposing over time. For example, throughout a decomposition life cycle, a polyester hydrogel may absorb and retain CO₂ received from the environment. A polyester hydrogel may also release CO₂ to surrounding plant life to serve as a carbon nutrient source.

By way of this reference the appended claims form an integral part of this disclosure.

### EXAMPLES

The following examples illustrate some specific example embodiments of the present disclosure. These examples represent specific approaches found to function well in the practice of the application, and thus can be considered to constitute examples of modes for its practice. However, those of skill in the art should, in light of the present disclosure, appreciate that many changes can be made in the specific embodiments that are disclosed without departing from the spirit and scope of the application.

Example 1

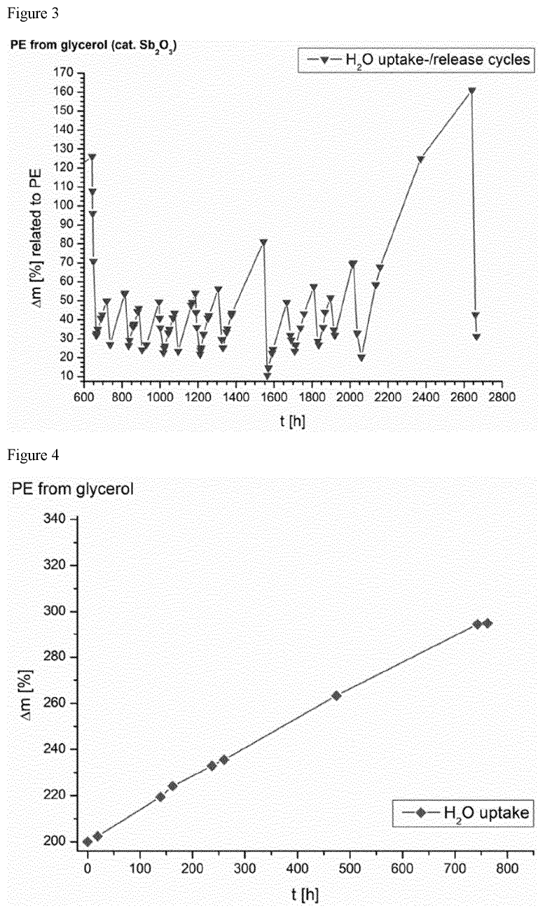

A polyester hydrogel was prepared from glycerin and catalytic (Sb₂O₃) according to disclosed embodiments and then contacted with water to analyze water uptake dependence on time with the results being shown in FIGS. 3 and 4. The hydrogel was introduced to a water source for 2,800 hours and the volume of the hydrogel was measured periodically. As shown in FIG. 3, the water uptake is dependent on time. Water was absorbed and released periodically and was shown to correlate with temperature. FIG. 3 shows the time dependence of the mass difference during water uptake/release cycles at alternating temperatures of 25° C. and 35° C. The volume change (Δm) ranged from 0 to about 160% throughout the 2,700 hour time frame. Additionally, as shown in FIG. 4, a hydrogel was able to absorb enough water to increase its mass to almost three times the original mass over an 800 hour period.

FIG. 4. The time dependence of the mass balance during water uptake of the sample at ambient temperature.

It is understood that the listed components for each unit are for illustration purposes only, and this is not intended to limit the scope of the application. A specific combination of these or other components or units can be configured in such a composition or method for the intended use based on the teachings in the application.

Persons skilled in the art may make various changes in the shape, size, number, separation characteristic, and/or arrangement of parts without departing from the scope of the instant disclosure. Each disclosed component, system, and process step may be performed in association with any other disclosed component, system, or process step and in any order according to some embodiments. Where the verb “may” appears, it is intended to convey an optional and/or permissive condition, but its use is not intended to suggest any lack of operability unless otherwise indicated. Persons skilled in the art may make various changes in methods of preparing and using a composition, device, and/or system of the disclosure. Where desired, some embodiments of the disclosure may be practiced to the exclusion of other embodiments.

Also, where ranges have been provided, the disclosed endpoints may be treated as exact and/or approximations as desired or demanded by the particular embodiment. Where the endpoints are approximate, the degree of flexibility may vary in proportion to the order of magnitude of the range. For example, on one hand, a range endpoint of about 50 in the context of a range of about 5 to about 50 may include 50.5, but not 52.5 or 55 and, on the other hand, a range endpoint of about 50 in the context of a range of about 0.5 to about 50 may include 55, but not 60 or 75. In addition, it may be desirable, in some embodiments, to mix and match range endpoints. Also, in some embodiments, each figure disclosed (e.g., in one or more of the examples, tables, and/or drawings) may form the basis of a range (e.g., depicted value +/− about 10%, depicted value +/− about 50%, depicted value +/− about 100%) and/or a range endpoint. With respect to the former, a value of 50 depicted in an example, table, and/or drawing may form the basis of a range of, for example, about 45 to about 55, about 25 to about 100, and/or about 0 to about 100.

These equivalents and alternatives along with obvious changes and modifications are intended to be included within the scope of the present disclosure. Accordingly, the foregoing disclosure is intended to be illustrative, but not limiting, of the scope of the disclosure as illustrated by the appended claims.

The title, abstract, background, and headings are provided in compliance with regulations and/or for the convenience of the reader. They include no admissions as to the scope and content of prior art and no limitations applicable to all disclosed embodiments.

1. A method for converting a CO₂ gas stream comprising a CO₂ into an ester, the method comprising:
converting the CO2 into a (COOH)2 preferably by passing the CO2 through a water bath to produce a carbonated water; and
passing the carbonated water through a metal ion exchange bubble column comprising a M₂(COO)₂ to produce the (COOH)₂ and a MHCO₃;
reacting the (COOH)₂ with a mono-alcohol to obtain the ester.

2. The method according to claim 1, further comprising:
(c) combining a glycerine, an acid catalyst comprising a H₂SO₄, and the ester to produce a hydrogel and a mono-alcohol,
wherein the hydrogel has a structure according to Formula I:

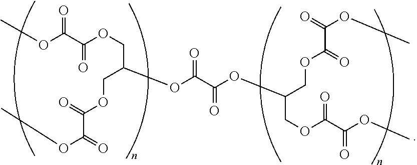

3. The method according to claim 1, wherein reacting the (COOH)₂ with a mono alcohol to obtain the ester comprises:
passing the (COOH)₂ and a mono-alcohol through an activated carbon bed to generate an ester;
wherein the ester comprises two or more of a (COOCH₃)₂ and a (COOEt)₂.

4. The method according to claim 1, further comprising:
combining a glycerine, an acid catalyst comprising a H₂SO₄, and the ester to produce a hydrogel and a mono-ethanol,
wherein the hydrogel has a structure according to Formula I:

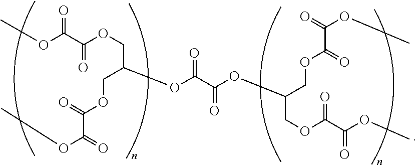

5. The method according to claim 4, further comprising:
combining the MHCO₃ produced from the metal ion exchange bubble column with a hydrogen gas in a hydrogenation reactor comprising a palladium catalyst at a temperature ranging from about 15° C. to about 100° C. and a pressure of about 0.1 bara to about 100 bara to produce a mixture comprising HCOOM and MHCO₃, wherein HCOOM comprises one or more of CHOOK and HCOONa.

6. The method according to claim 5, further comprising:
(e) separating the mixture through fractional crystallization in a crystallization unit into a separated MHCO₃ and a separated HCOOM.

7. The method according to claim 6, further comprising:
(f) feeding the separated MHCO₃ into the hydrogenation reactor.

8. The method according to claim 7, further comprising:
(g) treating the separated HCOOM with a catalytic amount of MOH at a temperature of ranging from about 100° C. to about 400° C. to produce a hydrogen gas and a dried M₂(COO)₂,
wherein MOH comprises one or more of NaOH, KOH and NH4OH and preferably KOH and/or NaOH, and
wherein the dried M₂(COO)₂ comprises one or more of K₂(COO)₂ and Na₂(COO)₂.

9. The method according to claim 8, further comprising at least one of:
transferring the hydrogen gas to the hydrogenation reactor; and
transferring the dried M₂(COO)₂ to the metal ion exchange bubble column.

10. A system for manufacturing an ester from a CO₂ gas stream comprising a CO₂, the system comprising:
a CO₂ conversion unit configured to convert the CO₂ into a (COOH)₂;
a metal ion exchange bubble column to produce the (COOH)₂ and a MHCO₃; and
a reactor for reacting the (COOH)₂ with a mono-alcohol to obtain an ester; preferably the reactor comprises an activated carbon bed configured to receive the (COOH)₂ and a mono-alcohol to generate the ester.

11. The system of claim 10, wherein the CO₂ conversion unit is configured to combine the CO₂ with a M₂(COO)₂ to produce a (COOH)₂ and a MHCO₃,
wherein the MHCO₃ comprises one or more of KHCO₃ and NaHCO₃, and
wherein M₂(COO)₂ comprises one or more of K₂(COO)₂ and Na₂(COO)₂.

12. The system of claim 10 further comprising a polymerization reactor configured to receive the ester and combine the ester with a glycerine and an acid catalyst comprising a H₂SO₄ to produce a polyester and an ethanol,
wherein the polyester has a structure according to Formula I:

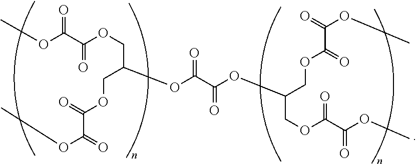

13. The system according to claim 10, further comprising:
(c) a hydrogenation reactor connected to the CO₂ conversion unit and configured to receive the MHCO₃ from the CO₂ conversion unit and to combine the MHCO₃ with a hydrogen gas and a palladium catalyst at a temperature ranging from about 35° C. to about 80° C. and a pressure ranging from about of 1 bara to about 30 bara to produce a mixture comprising MHCO₃ and HCOOM,
wherein HCOOM comprises one or more of HCOOK and HCOONa.

14. The system according to claim 13, further comprising:
(d) a crystallization unit configured to receive the mixture from the hydrogenation reactor and to separate the mixture through into a separated MHCO₃ and a separated HCOOM.

15. The system according to claim 14, further comprising:
(e) a dryer/inert treatment reactor configured to receive the separated HCOOM from the crystallization unit and to treat the separated HCOOM with a catalytic amount of KOH at a temperature of ranging from about 100° C. to about 400° C. to produce a hydrogen gas and a dried M₂(COO)₂,
wherein M₂(COO)₂ comprises a K₂(COO)₂ and Na₂(COO)₂.
